(19) United States
(12) Patent Application Publication
(10) Pub. No.: US 2022/0125648 A1
(43) Pub. Date: Apr. 28, 2022

(54) Moisture Absorbent Undergarment

(71) Applicant: Serenity Care Services, Inc., Chicago, IL (US)

(72) Inventor: Coprice Jones, Chicago, IL (US)

(73) Assignee: Serenity Care Services, Inc., Chicago, IL (US)

(21) Appl. No.: 17/079,114

(22) Filed: Oct. 23, 2020

(51) Int. Cl. A61F 13/47; A61F 13/62

(52) U.S. Cl. CPC A61F 13/47; A61F 2013/15146; A61F 13/62

(57) ABSTRACT

A liner for absorbing incontinently passed urine or other moisture passed at the crotch, underside, buttocks, and/or inner thigh area has a first extension, second extension, third extension each of which extend away from a liner intersection portion and each of which are connected thereto. The first extension inserts into a first pocket interior of a first pocket of a garment. The said second extension inserts into a second pocket interior of a second pocket of the garment. The third extension inserts into a third pocket interior of a third pocket of the garment. The fourth extension inserts into a fourth pocket interior of a fourth pocket of the garment. The first pocket comprises an interior anterior torso portion of the garment. The second pocket comprises an interior posterior torso portion of the garment. The third and fourth pocket comprise first and second interior inner thigh sections of the garment.

### I. FIELD OF THE INVENTION

The present disclosure concerns a crotch, underside, buttocks, and/or inner thigh disposed wearable liquid absorbing device. More particularly the device can include a plurality of delineated pocket interiors and a plurality of liner extensions received by the plurality of pocket interiors.

### II. BACKGROUND OF THE INVENTION

Devices for absorbing incontinently passed urine are known. U.S. Pat. No. 8,702,667, Johnson, concerns an undergarment defining an interior space and an exterior space. An aperture enables the penis of the wearer to extend through the aperture from the interior space to the exterior space. A flap at least partially covers the aperture and is selectively movable between an open position and a closed position. A pouch is located between the flap in the closed position and the undergarment. In a further example, a pad is configured to be placed within the pouch and confine the penis as it extends from the interior space of the undergarment, through the aperture, and into the pad. A method includes placing the undergarment about the body of the male incontinence garment wearer, placing the penis of the male incontinence garment wearer through the aperture and the hole into the pad.

US Patent Publication 2014/0378936, Coates, concerns an undergarment including a pocket extending from an anterior crotch portion of the undergarment to a posterior crotch portion of the undergarment, the pocket closed along three sides and opening in a direction of the anterior crotch, a hood arranged on the anterior crotch portion concealing the opening of the pocket, and an absorbent pad removable from the pocket, wherein the hood is configured to be pulled aside to expose the opening of the pocket to remove and insert the absorbent pad from the pocket.

US Patent Publication, 2013/0006209, Ruiz, concerns a two-piece wearer absorbent article having an outer cover and an absorbent insert is disclosed. The outer cover has a landing zone comprising a loop material disposed on the front waist region and extending laterally across substantially all the front waist region. The inner surface of the outer cover has an insert fastener component disposed in the front region thereof, and the insert fastener component is secured directly to the loop material.

### III. SUMMARY OF THE INVENTION

As an example, an object and/or advantage of the invention can include a liner for absorbing incontinently passed urine or other moisture passed at the crotch, underside, buttocks, and/or inner thigh area of a user. In the example, the liner includes an intersection portion. A first extension extends away from the liner intersection portion and connects thereto. The first extension has a first facing surface, a second facing surface, a proximal end at the intersection portion, a distal end spaced from said proximal end a distance. The distance can be measured along a first line traversing through first structure of the first extension wherein the first structure extends from the first extension proximal end to the first extension distal end. The first structure and first line reside between a first extension first side and a first extension second side.

The example further includes a second extension extending away from said liner intersection portion and connected thereto. The said second extension has a first facing surface, a second facing surface, a proximal end at the intersection portion, a distal end spaced from said proximal end a distance. The distance can be measured along a second line traversing through second structure of the second extension, wherein the second structure extends from the second extension proximal end to the second extension distal end. The structure and second line reside between a second extension first side and a second extension second side.

The example can also include a third extension of said liner extending away from said liner intersection portion and connected thereto. The third extension has a first facing surface, a second facing surface, a proximal end at the intersection portion, a distal end spaced from said proximal end a distance. The distance can be measured along a third line traversing through third structure of the third extension, wherein the third structure extends from the third extension proximal end to the third extension distal end. The structure and third line reside between a third extension first side and a third extension second side.

The example can further include a fourth extension of said liner extending away from said liner intersection portion and connected thereto. The fourth extension has a first facing surface, a second facing surface, a proximal end at the intersection portion, a distal end spaced from said proximal end a distance. The distance can be measured along a fourth line traversing through fourth structure of the fourth extension, wherein the fourth structure extends from the fourth extension proximal end to the fourth extension distal end. The structure and fourth line reside between a fourth extension first side and a fourth extension second side.

The example further includes the liner having an installed orientation, wherein in said installed orientation said first line comprises a first curve, the first curve extends along z, y coordinates of an x;−x, y;−y, z;−z coordinate system, said y, z coordinates change along the first curve; said second line comprises a second curve, the second curve extends along z,−y coordinates of an x;−x, y;−y, z;−z coordinate system, said −y, z coordinates change along the second curve; said third line comprises a third curve, the third curve extends along x,−z coordinates of an x;−x, y;−y, z;−z coordinate system, said x, −z coordinates change along the third curve; and said fourth line comprises a fourth curve, the fourth curve extends along −x,−z coordinates of an x;−x, y;−y, z;−z coordinate system, said −x, −z coordinates change along the fourth curve.

### IV. BRIEF DESCRIPTION OF THE DRAWINGS

FIG. 1 is a view of a female oriented liner of the present disclosure looking down onto a first facing surface of said liner.

FIG. 2 is a view of a male oriented liner of the present disclosure looking down onto a first facing surface of said liner.

FIG. 3A is an interior view of a female oriented garment of the present disclosure having the liner installed; said view looks down into the garment opened up.

FIG. 3B is an interior view of a male oriented garment of the present disclosure having the liner installed; said view looks down into the garment opened up.

FIG. 4A is the interior view of the female oriented garment of the present disclosure exclusive of the liner; said view looks down into the garment opened up.

FIG. 4B is the interior view of the male oriented garment of the present disclosure exclusive of the liner; said view looks down onto the garment opened as if worn by a user.

FIG. 5A is an exterior view of an anterior of the female garment of the present disclosure; said garment laid flat; said view looking down onto its anterior.

FIG. 5B is an exterior view of an anterior of the male garment of the present disclosure; said garment laid flat; said view looking down onto its anterior.

FIG. 6A is an interior view of an anterior of the female garment of the present disclosure; said garment laid flat and turned inside out; said view looking down into its anterior.

FIG. 6B is an interior view of an anterior of the male garment of the present disclosure; said garment laid flat and turned inside out; said view looking down onto its anterior.

FIG. 7A is an interior view of an anterior of the female garment of the present disclosure; said liner installed in said garment; and said garment laid flat and turned inside out; said view looking down onto its anterior.

FIG. 7B is an interior view of an anterior of the male garment of the present disclosure; said liner installed in said garment; and said garment laid flat and turned inside out; said view looking down onto its anterior.

FIG. 8A is a view of a female oriented liner of the present disclosure looking down onto a second facing surface of said liner.

FIG. 8B is a view of a male oriented liner of the present disclosure looking down onto a second facing surface of said liner.

FIG. 9A is an exterior view of a posterior of the female garment of the present disclosure; said garment laid flat; said view looking down onto its posterior.

FIG. 9B is an exterior view of a posterior of the male garment of the present disclosure; said garment laid flat; said view looking down onto its posterior.

FIG. 10A is an interior view of a posterior of the female garment of the present disclosure; said garment laid flat and turned inside out; said view looking down into its posterior.

FIG. 10B is an interior view of a posterior of the female garment of the present disclosure; said garment laid flat and turned inside out; said view looking down into its posterior.

FIG. 11A is an interior view of a posterior of the female garment of the present disclosure; said liner installed in said garment; and said garment laid flat and turned inside out; said view looking down onto its posterior.

FIG. 11B is an interior view of posterior of the male garment of the present disclosure; said liner installed in said garment; and said garment laid flat and turned inside out; said view looking down onto its posterior.

FIG. 12A is a generic view looking into a third surface of a pocket structure generic to all four pocket structures shown in the various figures.

FIG. 12B is a cross section of the generic pocket structure of FIG. 12A highlighting the structures three layers and coupling structures.

FIG. 12C is a cutout of a portion of liner looking into the liner's first layer; said liner generic to all five liner sections shown in the various figures.

FIG. 12D is a cross section of the generic liner shown in FIG. 12C highlighting the liner's three layers

FIG. 13 is a view of a female garment being worn by a female.

FIG. 14 is a view of a male garment being worn by a male.

### V. DETAILED DESCRIPTION OF THE INVENTION

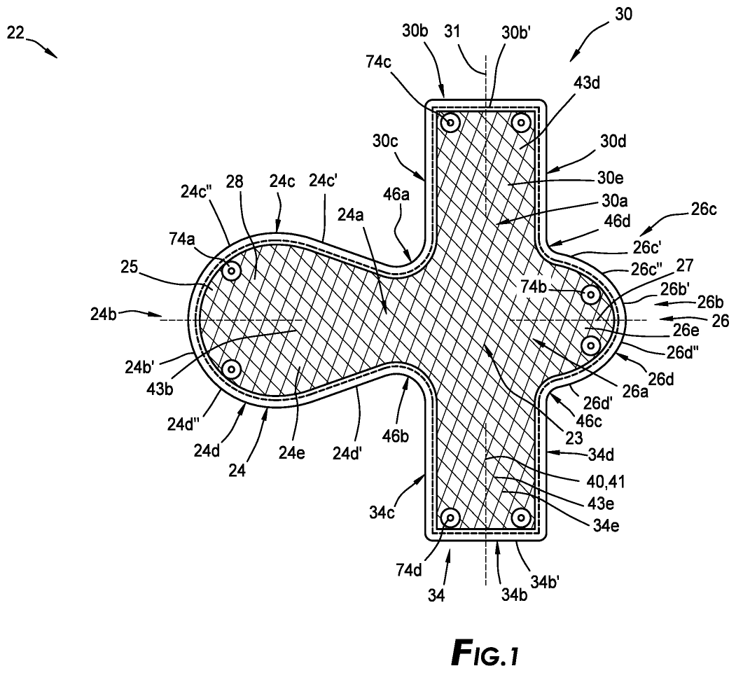

The invention concerns device(s) for absorbing incontinently passed urine or other moisture passed at the crotch, underside, buttocks, and/or inner thigh area of a user of the device. Now referring to FIGS. 1a, 1b; 2a, 2b, examples of the invention include a liner (22). The liner can comprise a pad, include padding or be a pad. The liner is reusable, hand and machine washable. The liner can have an intersection portion (23). A first extension (24) of the liner can extend away from the intersection portion (23) a distance. The distance is a first length measured along a first line (25) traversing through first structure of the first extension wherein the first structure extends from a first extension proximal end (24a) to a first extension distal end (24b), and the structure and first line (25) between a first extension first side (24c) and a first extension second side (24d). The first extension distal end can be spaced away from the first extension proximal the distance. The proximal end (24a) of the first extension (24) can merge with the intersection portion (23). It can end at the intersection portion. The first extension (24) can terminate at the distal end (24b). The distal end can be a free end of the first extension. The first extension (24) seamlessly connects with the intersection portion (23) and merges seamlessly with the intersection portion (23). The structure through which the first line (25) traverses can be continuous and seamlessly connected. The first extension first side (24c) and second side (24d) can be spaced apart. The sides can delimit side perimeters of the first extension (24). The sides extend away from the proximal end (24a) towards and to the distal end (24b). The sides can extend from the intersection portion (23). The sides can be straight and/or can be curved. In the present example each side can have a proximal portion (24c′, 24d′) which extends away from a medial portion of the first extension and a side distal portion (24c″, 24d″) which extends towards the medial portion of the first extension. The sides are continuously curvilinear. The medial portion being at the middle of a transverse distance between the first (24c) and second (24d) sides. Towards and at the first extension distal end (24b), each first (24c) and second (24d) side can merge into one another along a path (24b′). The path can be arcuate. Alternatively, the first extension distal end can be delineated along a substantially straight side that connects the first and second sides. In any event the first and second sides are connected at the distal end. In the present example the sides merge into each other along an arcuate path.

Relative to spatial coordinates x;−x and y;−y and z;−z the first line (25) traversing through the first structure of the first extension (24) can be straight and/or can be curved. The shape and trajectory of the first line (25) and the structure through which it traverses can change based on whether the liner is in an installed orientation or an uninstalled orientation. In the installed orientation the structure through which the first line traverses can extend along a plane. The first line and the structure through which it traverses along the plane can have an x;−x coordinate which remains constant. The y;−y and the z;−z coordinate of the first line and the structure through which it traverses along the plane can change. In the installed orientation, first line and the structure through which it traverses along the plane can each comprise a curve. The curves can be the same. The curve can intersect a single x;−x coordinate. In the shown embodiment, in the installed orientation, the x coordinate can be zero. The y and z coordinates change along the curve. It is possible in the installed and uninstalled orientation the x; −x and −y; y and z; −z coordinates of the first line and the structure through which it traverses could change. In the uninstalled orientation, the liner can be laid flat and then the first line and the structure through which it traverses can be straight and can change only along the y coordinate. In the shown embodiment the first line and the structure through which it traverses, in the uninstalled orientation, extend and changes in the y direction.

A second extension (26) of the liner can extend away from the intersection portion (23) a distance. The distance is a second length measured along a second line (27) traversing through second structure of the second extension wherein the second structure extends from a second extension proximal end (26a) to a second extension distal end (26b), and the structure and second line between a second extension first side (26c) and a second extension second side (26d). The second extension distal end (26b) can be spaced away from the second extension proximal end the distance (26a). The proximal end (26a) of the second extension can merge with the intersection portion (23). It can end at the intersection portion (23). The second extension can terminate at the distal end (26b). The distal end can be a free end of the second extension (26). The second extension (26) seamlessly connects with the intersection portion (23) and merges seamlessly with the intersection portion (23). The structure through which the second line (27) traverses can be continuous and seamlessly connected. The second extension first side (26c) and second side (26d) can be spaced apart. The sides can delimit side perimeters of the second extension. The sides extend away from the proximal end (26a) towards and to the distal end (26b). The sides can extend from the intersection portion (23). The sides can be straight and/or can be curved. In the present example for the male oriented device, each side can have a proximal portion (26c′, 26d′) which extends away from a medial portion of the second extension and a side distal portion (26c″, 26d″) which extends towards the medial portion of the second extension. The sides are continuously curvilinear. In the female oriented device, side (26c) and side (26d) are arcuate and each curve to the medial portion. The sides are continuously curvilinear. The medial portion being at the middle of a transverse distance between the first and second sides. Towards and at the second extension distal end, each first and second side can merge into one another along a path. The path can be arcuate. Alternatively, the second extension distal end can be delineated along a substantially straight that connects the first (26c) and second (26d) sides. In any event the first and second sides are connected at the distal end (26b). In the present male oriented example, the sides are connected along straight side (26b′). In the female oriented example, the sides (26c) and (26d) merge along and arcuate path (26b′).

Relative to spatial coordinates x; −x and y;−y and z;−z the second line (27) traversing through the second structure of the second extension can be straight and/or can be curved. The shape and trajectory of the second line and the second structure through which it traverses can change based on whether the liner is in an installed orientation or an uninstalled orientation. In the installed orientation the structure through which the second line (27) traverses can extend along a plane. The second line (27) and the structure through which it traverses along the plane can have an x;−x coordinate which remains constant. The y;−y and the z;−z coordinate of the second line (27) and the structure through which it traverses along the plane can change. In the installed orientation, second line and the structure through which it traverses along the plane can each comprise a curve. The curves can be the same. The curve can intersect a single x;−x coordinate. In the shown embodiment, in the installed orientation, the x coordinate can be zero. The −y and z coordinates change along the curve. It is possible in the installed and uninstalled orientation the x; −x and −y; y and z; −z coordinates of the second line (27) and the structure through which it traverses could change. In the uninstalled orientation, the liner can be laid flat and then the second line 27 and the structure through which it traverses can be straight and can change only along the −y coordinate. In the shown embodiment the second line (27) and the structure through which it traverses, in the uninstalled orientation, extend and changes in the −y direction.

Preferably the first extension (24) and second extension (26) when laid flat extend in opposite directions and along a first axis (28). The first sides of the first and second extension lay along a same plane. Also, the second sides of the first and second extension lay along a same plane. The first and second extension can be symmetrical along the first axis (28). The intersection portion (23) can also be symmetrical along the first axis (28). When laid flat the first axis extends in a longitudinal direction.

A third extension (30) of the liner can extend away from the intersection portion a distance. The distance is a third length measured along a third line (31) traversing through third structure of the third extension wherein the third structure extends from a third extension proximal end (30a) to a third extension distal end (30b), and the structure and third line (31) between a third extension first side (30c) and a third extension second side (30d). The third extension distal end (30b) can be spaced away from the third extension proximal end (30a) the distance. The proximal end (30a) of the third extension can merge with the intersection portion (23). It can end at the intersection portion (23). The third extension can terminate at the distal end (30b). The distal end (30b) can be a free end of the third extension (30). The third extension seamlessly connects with the intersection portion and merges seamlessly with the intersection portion (23). The structure through which the third line (31) traverses can be continuous and seamlessly connected. The third extension (30) first side (30c) and second side (30d) can be spaced apart. The sides can delimit side perimeters of the third extension. The sides extend away from the proximal end (30a) towards and to the distal end (30b). The sides can extend from the intersection portion (23). The sides can be straight and/or can be curved. In the present example each side can have portions parallel to the other side. The portions can be majority portions of each side. Towards and at the third extension distal end (30b), each first and second side can merge into one another along a path. The path can be arcuate. Alternatively, the third extension distal end can be delineated along a substantially straight side (30b′) that connects the first and second sides. In any event the first and second sides are connected at the distal end. In the present example the distal end has a side (30b′) delineated along a substantially straight line that connects the first and second sides.

Relative to spatial coordinates x; −x and y;−y and z;−z the third line (31) traversing through the third structure of the third extension (30) can be straight and/or can be curved. The shape and trajectory of the third line (31) and the third structure through which it traverses can change based on whether the liner is in an installed orientation or an uninstalled orientation. In the installed orientation the third structure through which the third line (31) traverses can extend along a plane. The third line (31) and the third structure through which it traverses along the plane can have an y;−y coordinate which remains constant. The x;−x and the z;−z coordinate of the third line (31) and the third structure through which it traverses along the plane can change. In the installed orientation, the third line (31) and the structure through which it traverses along the plane can each comprise a curve. The curves can be the same. The curve can intersect a single y;−y coordinate. In the shown embodiment, in the installed orientation, the y coordinate can be zero. The x and −z coordinates change along the curve. It is possible in the installed and uninstalled orientation the x; −x and −y; y and z; −z coordinates of the third line 31 and the third structure through which it traverses could change. In the uninstalled orientation, the liner can be laid flat and then the third line 31 and the third structure through which it traverses can be straight and can change only along the x coordinate. In the shown embodiment the third line (31) and the third structure through which it traverses, in the uninstalled orientation, extend and changes in the x direction.

A fourth extension (34) of the liner can extend away from the intersection portion (23) a distance. The distance is a fourth length measured along a fourth line (40) traversing through fourth structure of the fourth extension wherein the fourth structure extends from a fourth extension proximal end (34a) to a fourth extension distal end (34b), and the fourth structure and fourth line (40) between a fourth extension first side and a fourth extension second side. The fourth extension distal end (34b) can be spaced away from the fourth extension proximal end (34a) the distance. The proximal end (34a) of the fourth extension can merge with the intersection portion (23). It can end at the intersection portion (23). The fourth extension can terminate at the distal end (34b). The distal end (34b) can be a free end of the fourth extension (34). The fourth extension (34) seamlessly connects with the intersection portion (23) and merges seamlessly with the intersection portion (23). The structure through which the fourth line (40) traverses can be continuous and seamlessly connected. The fourth extension first side (34c) and second side (34d) can be spaced apart. The sides can delimit side perimeters of the fourth extension. The sides extend away from the proximal end (34a) towards and to the distal end (34b). The sides can extend from the intersection portion (23). The sides can be straight and/or can be curved. In the present example each side can have portions parallel to the other side. The portions can be majority portions of each side. Towards and at the fourth extension distal end (34b), each first (34c) and second side (34d) can merge into one another along a path. The path can be arcuate. Alternatively, the fourth extension distal end (34b) can be delineated along a substantially straight side (34b′) that connects the first and second sides. In any event the first and second sides are connected at the distal end. In the present example the distal end has a side (34b′) delineated along a substantially straight line.

Relative to spatial coordinates x; −x and y;−y and z;−z the fourth line (40) traversing through the fourth structure of the fourth extension can be straight and/or can be curved. The shape and trajectory of the fourth line (40) and the structure through which it traverses can change based on whether the liner (22) is in an installed orientation or an uninstalled orientation. In the installed orientation the fourth structure through which the fourth line (40) traverses can extend along a plane. The fourth line (40) and the fourth structure through which it traverses along the plane can have an y;−y coordinate which remains constant. The x;−x and the z;−z coordinate of the fourth line (40) and the fourth structure through which it traverses along the plane can change. In the installed orientation, fourth line (40) and the fourth structure through which it traverses along the plane can each comprise a curve. The curves can be the same. The curve can intersect a single y;−y coordinate. In the shown embodiment, in the installed orientation, the y coordinate can be zero. The −x and −z coordinates change along the curve. It is possible in the installed and uninstalled orientation the x; −x and −y; y and z; −z coordinates of the fourth line (40) and the fourth structure through which it traverses could change. In the uninstalled orientation, the liner can be laid flat and then the fourth line (40) and the fourth structure through which it traverses can be straight and can change only along the −x coordinate. In the shown embodiment the fourth line and the fourth structure through which it traverses, in the uninstalled orientation, extend and change in the −x direction.

When laid flat, the third (30) and fourth (34) extensions are symmetrical along a second axis (41). The second axis extends through the proximal (30a, 34a) and distal (30b, 34b) ends of each of the third (30) and fourth (34) extension. The extensions extend in opposite directions. The second axis (41) is a transverse axis extending in the transverse direction.

Each of the first (24), second (26), third (30) and fourth (34) liner extensions and the liner intersection portion (23) have a respective first facing surface (23e, 24e, 26e, 30e, 34e) oppositely facing from its second facing surface (23f, 24f, 26f, 30f, 34f). Each first facing surface can comprise a liquid absorbing material. Each first facing surface can comprise a series of depressions (35) each of which are depressed areas. The surface can comprise surface of padding.

In more detail, the above described liner (22), including its extensions (24, 26, 30, 34) and intersection portion (23), each have a plurality of layers. The liner and each of its extensions and its intersection portion can have a first layer (43a, 43b, 43c, 43d, 43e) which comprises liquid absorbent material. The first layer also comprises padding. The first layer has the first facing surface (23e, 24e, 26e, 30e, 30f). The first layer can comprise the series of depressions (35) each of which are depressed areas.

The liner, its extensions and intersection portion can have a second layer (44a, 44b, 44c, 44d, 44e) made of liquid impermeable or resistant or repellent material. The second layer (44a-44e) can be over, opposite, opposed to and overlapping the first layer (43a-43e) and proximate thereto. More particularly it can be over, opposite, opposed to and overlapping and proximate a second facing surface of the first layer (43a-43e); the second facing surface is on an opposite side of the first layer (43a-43e) than is the first facing surface (23e, 24e, 26e, 30e, 34e). The second facing surface faces in a direction opposite the first facing surface. The second layer (44a-44e) is coupled to the first layer (43a-43e). The liner, its extensions and intersection portion have a third layer (45a-45e). The second layer (44) is between the third layer (45) and the first layer (43). The third layer (45) can have a material which is the same as the material forming the garment's thigh sections and torso sections. The third layer comprises second facing surfaces (23f, 24f, 26f, 30f, 34f) of the liner. The third layer can be or have a coating thereon which can be liquid impermeable, repellent and resistant. The second facing surface can also be or have a coating thereon which can be liquid impermeable, repellent and resistant.

The liner includes a plurality of junctions. One junction (44a) of said plurality can be where first sides (24c, 30c) of the first (24) and third (30) extensions are joined to each other or are connected to each other. The first sides (24c, 34c) are adjacent and delimit a space there between. A second junction (44b) can be where the second side (24d) of the first extension (24) and first side (34c) of the fourth extensions (34) are joined to each other or are connected to each other. The second and first sides are adjacent and delimit a space there between. A third junction (46c) can be where second sides (26d, 34d) of the second (26) and fourth (34) extensions are joined to each other or are connected to each other. The second sides are adjacent and delimit a space there between. A fourth junction (46d) can be where a first side (26c) of the second extension (26) and second side (30d) of the third extension (30) are joined to each other or are connected to each other. The second and first sides are adjacent and delimit a space there between.

The interior portion (47) of the garment itself has an interior facing surface (48) delimiting interior of the garment. The garment can be an undergarment as shown. The interior portion of the garment includes all structure delimiting a pocket interior, forming a pocket structure, and/or within the garments exterior surface (49). The interior portion has an interior crotch portion (50′) which is over, opposite, opposed to and overlaps a crotch portion of a user when the garment is worn by the user. The interior portion also has an interior buttocks portion (51′) which is over, opposite, opposed to and overlaps the buttocks when the garment is worn by a user. The interior portion also has an interior of an underside portion (52) which can be over, opposite, opposed to and overlapping an underside of the user between the user's crotch and the first end of the user's crevice. The first end being the end of the crevice opposite an end of the crevice proximate the tail bone.

The garment can have a torso portion (53). The torso portion has an upper section (53a) which extends about a person's torso and delimits an opening into the interior (47) of the garment when the garment is worn by a user. The upper section (53a) can comprise a waist band. The band can be elastic. The upper section (53a) has an interior surface which can be over, opposite, opposed to and overlaps a user's torso when the garment is worn by a user. The upper section (53a) can extend around the torso above the bellybutton, at the bellybutton, just below the bell button or at the waist of a user. In the female oriented device, a hemmed under fold forms the upper section (53a) and can be considered a band. The band includes the same material as the garment anterior and posterior torso sections (53b) and (53c). In the male oriented device, the upper section (53a) is an elastic band of distinct material different form said posterior and anterior torso sections. The garment torso portion (53) includes the anterior torso section (53b) which includes the crotch portion and the upper section. It is between the upper section and the underside. The anterior torso section portion has an inner surface which delimits an interior (53b′) of the anterior torso section (53b). It can be over, opposite, opposed to and overlap the torso above the bellybutton, at the bellybutton, below the belly button, and/or the lower abdomen when a user wears the garment. The torso section includes the posterior torso section (53c) which is between the upper section and the underside. It includes the buttocks portion (51). The posterior torso section has an interior surface which delimits an interior (53c′) of the posterior torso section (53c). It can be over, opposite, opposed to and overlap the tail bone and can be over, opposite, opposed to and overlap the lower back when a user wears the garment. The buttocks portion has an interior surface (51′) which can be over, opposite, opposed to and overlap the tail bone and can be over, opposite, opposed to and overlap the buttocks.

The garment has first (54) and second (55) leg portions. Each leg portion has an opening delimiting section (54a, 55a) which extends about a person's leg and delimits an opening through which a respective leg extends when the garment is worn by a user. Each opening opens into an interior of the garment. Each opening delimiting section can comprise a band. The band can be an elastic band. The band can comprise a hemmed under or over fold formed from garment material. Each band can have an internal layer which is water impermeable, resistant or repellent. The layer can be a thin coating. Each leg portion has a thigh section (54b, 55b). The leg opening delimiting portion can have an interior surface opposite, over and overlapping the thigh of a user. A user is a wearer of the garment. The inner surface of the opening delimiting portion can be or include liquid impermeable, resistant, or repellent material. The material can be a coating which can be a thin film. Each thigh section can extend between its respective leg opening delimiting section (54a, 55a) on the one hand and the garment underside portion (52), and the garment's buttocks portion (51) on the other. Each thigh section (54b, 55b) extends from the garment underside (52) to its respective leg portion's opening delimiting section (54a, 55a). Each thigh section can extend about a respective thigh of a user when worn. Each thigh section (54b, 55b) has an inner surface which delimits an interior of the garment thigh section. Each interior thigh section (54b′, 55b′) can be over, opposite, opposed to and overlapping a respective thigh when the garment is worn. Each interior thigh section has an inner thigh portion (54c, 55c) which can extend over, opposite, opposed to and overlaps a respective inner thigh when worn. Each interior thigh section (54b′, 55b′), including its inner thigh portion (54c, 55c), can extend a distance of a traditional brief style boxer along the length of its respective thigh, including the inner thigh, when worn. Each interior thigh section (54b′, 54b′), including its inner thigh portion (55c), can extend at least a few inches from the interior garment underside to its respective opening delimiting section.

The garment's leg portions, torso portion, including the buttocks and crotch portions, can include a liquid impermeable, resistant or repellent layer forming the exterior surface (49) of the garment. The layer can be a thin coating. The material forming the leg portions, torso portion, including the buttocks and crotch portions, can be form fitting elastic material. The female oriented device can include an elastic lace outward most portion over elastic layers forming the leg portions, torso portion, including the buttocks and crotch portions. Both the male and female garments can be form fitting and can comprise form fitting elastic fabric.

The first extension (24), second extension (26) and intersection portion (23) of the liner are each over, opposite, opposed to and overlap interior facing surfaces (48) of the garment interior portion (47) when in an installed orientation. When the liner (22) is in an installed orientation, the first extension (24) is over, opposite, opposed to and overlaps the garment's interior crotch portion (50′). A second facing surface (24f) of the first extension (24) faces the interior facing surface of the interior crotch portion (50′). The first extension (24) can be considered a crotch portion of the liner. In the installed orientation, the first line (25) and first structure through which it traverses can be curved as explained above.

The liner first extension (24) and its second facing surface (24f), when in an installed orientation, can extend from the liner intersection portion (23) to be over, opposite, opposed to and overlap an interior (53b′) of other garment anterior torso sections (53b) in addition to the interior crotch portion (50′) of the garment. More particularly the first extension (24) and its second surface (24f) can be over, opposite, opposed to and overlap the anterior torso section (53b) located just below the bellybutton or at the bellybutton. It can be located one inch below the bellybutton. The first extension (24) and its first facing surface (24e) can be over, opposite, opposed to and overlapping the crotch of a user and other interior anterior torso sections of a user when the liner is in an installed orientation and the garment is worn by a user.

When the liner (22) is in the installed orientation, the second extension (26) is over, opposite, opposed to and overlaps the garment's interior buttocks portion/surface (51′). A second facing surface (26f) of the second extension (26) faces the interior surface of the interior buttocks portion (51′). The second extension can be considered a buttocks portion. The second line (27) and second structure through which it traverses can be curved as explained above.

The liner second extension (26) and its second facing surface (26f), when in an installed orientation, can extend from the liner intersection portion (23) to be over, opposite, opposed to and overlap other interior (53c′) garment posterior torso sections (53c) in addition to the interior buttocks portion (50′). More particularly the second extension and its second surface can be over, opposite, opposed to and overlap the interior garment posterior torso section (53c′) just below the tail bone. The second extension (26) and its first facing surface (24e) can be over, opposite, opposed to and overlapping the buttocks of a user and other interior posterior torso sections when the liner is in an installed orientation and the garment is worn by a user.

When the liner is in the installed orientation, the liner's intersection portion (23) is over, opposite, opposed to and overlaps the garment's interior underside portion (52) of the interior portion (47). The liner intersection portion (23) can be considered an underside portion. The intersection portion (23) and its first facing surface (23e) can be over, opposite, opposed to and overlapping the underside of a user when the liner is in an installed orientation and the garment is worn by a user. In an installed orientation, the liner (22), as explained more fully below, is removably coupled to the garment.

When the liner is in the installed position the third extension (30) and its second facing surface (30f) is over, opposite, opposed to and overlaps the inner surface of the garment interior first thigh section (54b′). The third extension (30) and its second facing surface (30f) is over, opposite, opposed to and overlaps the inner thigh portion (55c) of the garment first thigh section and extends in a direction away from said interior of said garment underside (52) towards said leg portion opening delimiting section (54a). The structure can extend at least a few inches towards the opening delimiting portion (54a). The structure can extend to a point where its distal end terminates proximate its leg portion's opening delimiting section (54a). The structure can extend without doubling back. The third extension (30) can be called a first inner thigh portion. The third extension (30) and its first facing surface (30e) can be over, opposite, opposed to and overlapping the first inner thigh of a user when the liner is in an installed orientation and the garment is worn by a user.

When the liner is in the installed position the fourth extension (34) and its second facing surface (34f) is over, opposite, opposed to and overlaps the inner surface of the garment interior second thigh section (55b′). The fourth extension (34) is over, opposite, opposed to and overlaps the inner thigh portion (55c) of the second interior thigh section (55b′) and extends in a direction away from said interior of said garment underside (52) towards said leg portion's opening delimiting section (55a). The structure can extend at least a few inches towards the opening delimiting section (55a). It can extend to a point where its distal end terminates proximate its leg portion's opening delimiting section (55a). The structure can extend without doubling back. The fourth extension (34) can be called a second inner thigh portion. The fourth extension (34) and its first facing surface (34e) can be over, opposite, opposed to and overlapping the second inner thigh of a user when the liner is in an installed orientation and the garment is worn by a user.

Each respective liner first facing surface (23e, 24e, 26e, 30e, 34e) can extend over, be opposite, opposed to and overlapping, one of a different one of a plurality of garment internal surfaces (60a′, 64a′, 66a′, 68a′); each of the plurality of garment internal surfaces each delimit a pocket interior (60c, 64c, 66c, 68c) of a different one of a plurality of pockets.

Each garment internal surface (60a′, 64a′, 66a′, 68a′); delimiting its respective pocket interior (60c, 64c, 66c, 68c) can be formed from a first layer (60a, 64a, 66a, 68a) of a respective interior multilayer structure (70a-70b). Each multilayered structure is a pocket stricture. It can of course be a single layer or a double layer. The structure can form part of the interior garment portion (47). The first layer (60a, 64a, 66a, 68a) and its internal surface (60a′, 64a′, 66a′, 68a′) can be liquid permeable. The first layer internal surface can also be or have a coating thereon which can be liquid impermeable, repellent and resistant. Each multilayer structure (70a-70d) can have a second layer (60b, 64b, 66b, 68b). The second layer can be liquid impermeable, repellent and resistant. The second layer (60b, 64b, 66b, 68b) is over, opposite, opposed to and overlapping the first layer (60a, 64a, 66a, 68a). Each multilayer structure (70a-70d) can have a third layer (60d, 64d, 66d, 68d). The second layer can be between the third layer and first layer. Each respective first layer (60a, 64a, 66a, 68a) can have a respective coupling structure (72a;72d) at the first layer and more particularly in the pocket interior (60c, 64c, 66c, 68c) and more particularly at each internal surface (60a′, 64a′, 66a′, 68a′). Each coupling structure can be a portion of a fastener such as a portion of snap(s). It can also be either the hooks or loops of a hook and loop fastener structure. The coupling structure can also comprise adhesive. The second layer (60b, 64b, 66b, 68b) and/or third layer (60d, 64d, 66d, 68d) shields any portion of the coupling structure from the skin of a user when the user wears the garment. For instance, the layers would shield the skin from a portion of the coupling at a surface of the first layer, wherein the surface is oppositely facing from the first layer's internal surface (60a′64a′, 66a′, 68a′).

The internal surface of each multilayer structure delimits an interior (60c, 64c, 66c, 68c) of its respective pocket. The interior of each pocket can be called a pocket interior, and each pocket interior can comprise a pocket void space. Each multilayer structure is connected to a portion of the garment when the liner (22) is in the installed position. As shown, each layer (60a, 64a, 66a, 68a) having the internal surface (60a′, 64a′, 66a′, 68a′) and each multilayered structure (70a-70d) can be coupled to a garment portion internal surface independently of the liner and can be coupled when the liner is not installed. The coupling can be with stitches or adhesive or other means. Each multilayered structure (70a-70d) having the internal surface (60a′, 64a′, 66a′, 68a′), comprises an end portion (71a-71d) which delimits an opening (71a-71d) into its respective pocket interior (60c, 64c, 66c, 68c). Each multilayered structure (70a-70d) is a pocket structure (70a-70d).

The end portions (71a-71b) are spaced apart from each other a distance along a longitudinal length taken along a first axis (28) measured a distance at least 25% and more usually at least 50% of the length of the garment interior underside (52).

A first one (60a′) of the plurality of the internal surfaces delimiting one of the plurality of the pocket interiors (60c), can be itself be over a surface. The surface which it is over, opposite, and opposed to can be a portion of the interior surface which delimits the interior garment crotch portion (50′) and/or a portion of the interior surface (53b′) of other garment anterior torso sections (53b). The portion can be a water impermeable, resistant or repellent layer. The layer can be a thin coating. In the shown embodiments, the structure which forms the interior surface (53b′) of the anterior torso section (53b) and/or the interior crotch portion (50′) further delimits a first one of pocket interiors (60c). The further delimiting structure can be or have a coating thereon which can be liquid impermeable, repellent and resistant. The first layer internal surface can also be or have a coating thereon which can be liquid impermeable, repellent and resistant. The distal end (24b) of the first extension (24) is in the first one of the pocket interiors (60c). The first extension first facing surface (24e) is over, opposite, opposed to, and overlapping the internal surface (60a′) of the first multilayered structure (70a) which is a first pocket structure. The first extension (24) has a coupling structure (74a) which releasably mates with the coupling structure (72a) at the internal surface (60a′) of the first multilayer structure (70a). The mating can form a releasable coupling of the first extension (24) and the multilayered structure, first pocket structure (70a). The first extension coupling structure (74a) is at the extension's first facing surface (24e) and preferably more towards, adjacent and proximate the distal end (24b).

A second one (64a′) of the plurality of the internal surfaces delimiting one of the plurality (64c) of pocket interiors is itself over a surface. The surface which it is over, opposite, and opposed to can be the interior surface which delimits the interior (53c′) of the garment posterior torso section (53c) and/or including the interior buttocks portion (51′). The portion can be a water impermeable, resistant or repellent layer. The layer can be a thin coating. In the shown embodiments, the structure which forms the interior surface (53c′) of the posterior torso section (53c) and/or including the interior buttocks portion (51′) further delimits a second one (64c) of the pocket interiors. The further delimiting structure can be or have a coating thereon which can be liquid impermeable, repellent and resistant. The first layer internal surface can also be or have a coating thereon which can be liquid impermeable, repellent and resistant. The distal end (26b) of the second extension (26) is in the second one (64c) of the pocket interiors. The second extension first facing surface (64e) is over, opposite, opposed to and overlapping the internal surface (64a′) of the second multilayered structure (70b). The second multilayered structure can be called a second pocket structure (70b). The second extension (26) has a coupling structure (74b) which releasably mates with the coupling structure (72b) on the second internal surface (64a′) of the second pocket structure (70b). The mating can form a releasable coupling of the second extension (26) and the second multilayered structure (70b). The second extension coupling structure (74b) is at the second extension's first facing surface (26e) and preferably more towards, adjacent and proximate the distal end (26b).

A third one (66a′) of the plurality of the internal surfaces delimiting one of the plurality (66c) of pocket interiors, is itself over a surface. The surface which it is over, opposite, and opposed to can be the interior surface which delimits the inner thigh portion (54c) of the first thigh section (54b). The portion can be a water impermeable, resistant or repellent layer. The layer can be a thin coating. In the shown embodiments, the structure which forms the inner thigh portion (54c) further delimits the third one of the pocket interiors (66c). The further delimiting structure can be or have a coating thereon which can be liquid impermeable, repellent and resistant. The first layer internal surface can also be or have a coating thereon which can be liquid impermeable, repellent and resistant. The distal end (30b) of the third extension (30) is in the third pocket interior (66c). The third extension first facing surface (66e) is over, opposite, opposed to and overlapping the internal surface (66a′) of the third multilayered structure (70c). The third multilayer structure can be called a third pocket structure (70c). The third extension (30) has a coupling structure (74c) which releasably mates with the coupling structure (72c) on the internal surface (66a′) of the third multilayered structure (70c). The mating can form a releasable coupling of the third extension (30) and the third pocket structure (70c). The third extension coupling portion (74c) is at the extension's first facing surface (30e) and preferably more towards, proximate and adjacent the distal end (30b).

A fourth one (68a′) of the plurality of the internal surfaces delimiting one of the plurality (68c) of pocket interiors, is itself over a surface. The surface which it is over, opposite, and opposed to can be the interior surface which delimits the inner thigh portion (55c) of the second thigh section (55b). The portion can be a water impermeable, resistant or repellent layer. The layer can be a thin coating. In the shown embodiments, the structure which forms the inner thigh portion (55c) further delimits the fourth one (68c) of the pocket interiors. The further delimiting structure can be or have a coating thereon which can be liquid impermeable, repellent and resistant. The first layer internal surface can also be or have a coating thereon which can be liquid impermeable, repellent and resistant. The distal end (34b) of the fourth extension (34) is in the pocket interior (68c). The fourth extension first facing surface (34e) is over, opposite, opposed to and overlapping the internal surface (68a′) of the fourth multilayered structure (70d) which can be called a fourth pocket structure (70d). The fourth extension (30) has a coupling structure (74d) which releasably mates with the coupling structure (72d) on the internal surface (68a′) of fourth multilayered structure (70d). The mating can form a releasable coupling of the fourth extension (30) and the fourth pocket structure (70d). The fourth extension coupling structure (74d) is at the extension's first facing surface (30e) and preferably more towards, proximate and adjacent the distal end (30b).

As explained above each first surface of each extension can have coupling structure. The coupling structure can be a portion of a fastener structure such as a portion of snap(s). It can also be either the hooks or loops of a hook and loop fastener structure. It can further be either a catch or a latch of a catch and latch fastener structure. The coupling structure can also comprise adhesive.

Each first facing surface of the liner, including each extension section and the intersection section, is over, opposite, and overlaps a body portion when the liner is installed in the garment and the garment is worn. The first extension first surface is over, opposite, and overlaps the anterior torso portion including the crotch portion. The second extension first surface is over, opposite, and overlaps the posterior torso portion including the buttocks portion. The third extension first surface is over, opposite, and overlaps the inner thigh portion. The fourth extension first surface is over, opposite, and overlaps the inner thigh portion. The intersection section first surface is over, opposite, and overlaps the user underside. When the garment and/or liner is described as overlapping a body portion the garment and liner is in the worn orientation.

The above written description, except where distinguished, generally reads on both the male and female oriented devices.

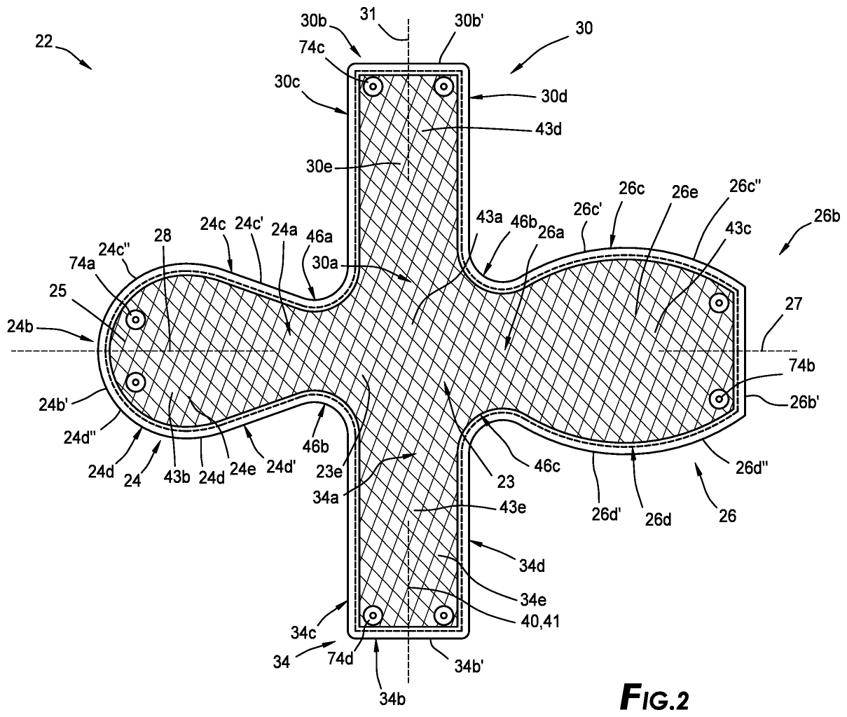

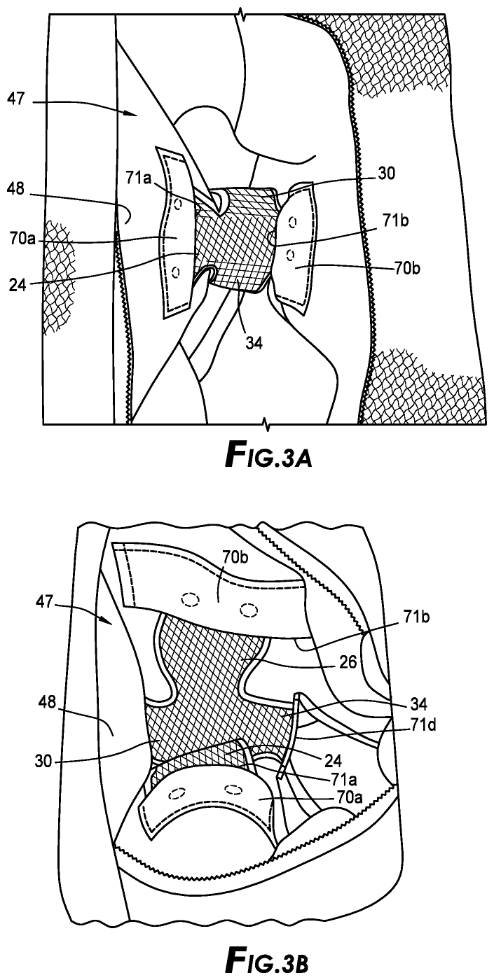

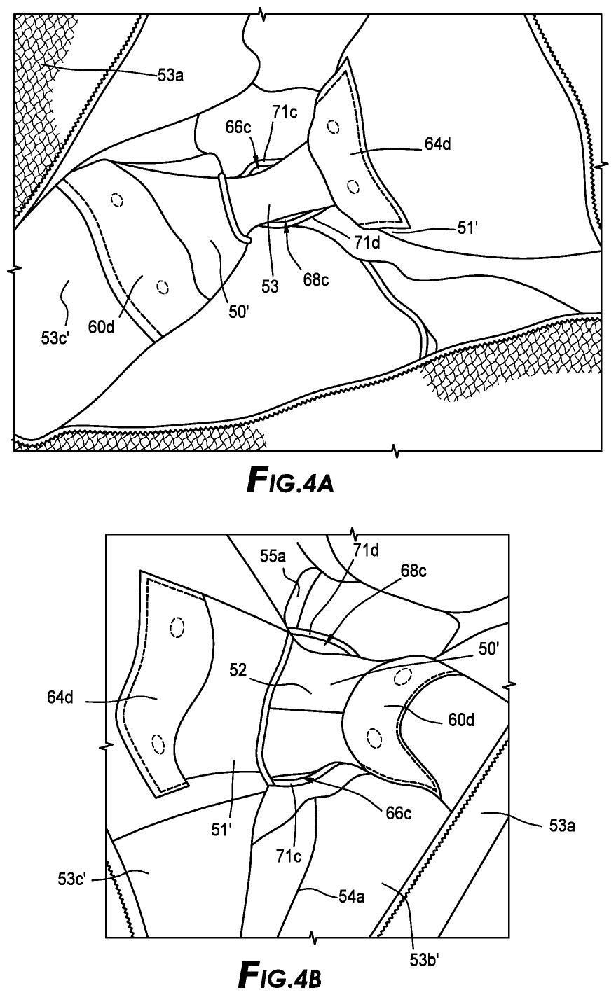

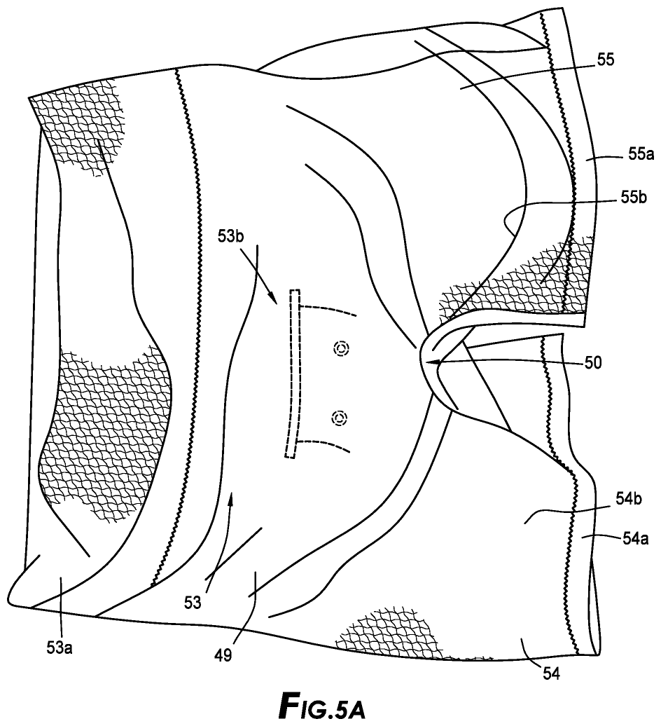

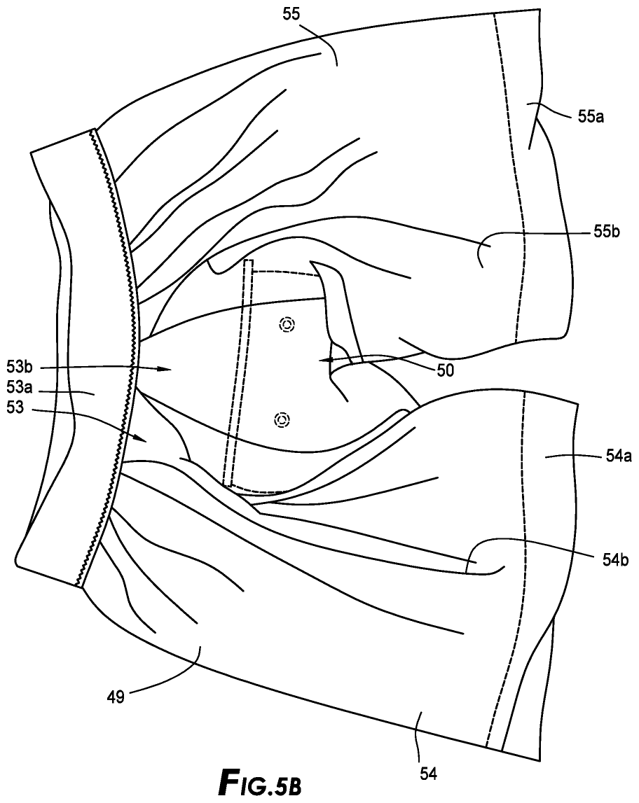

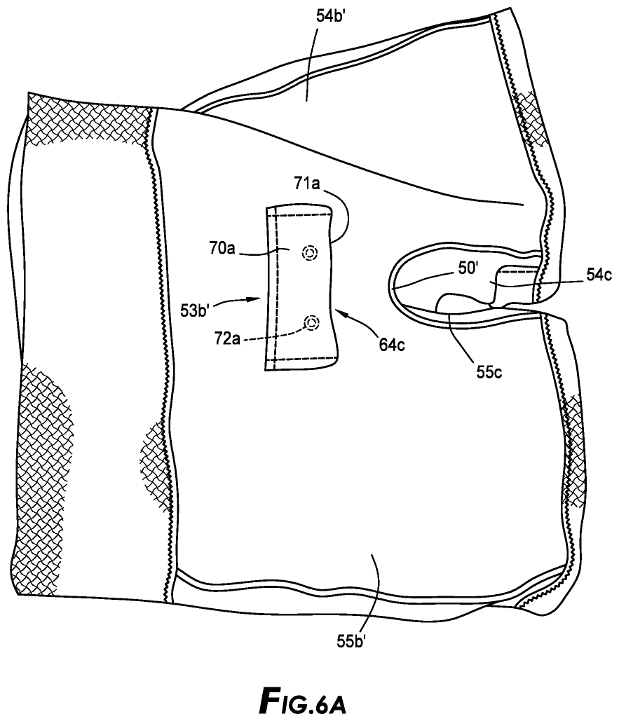

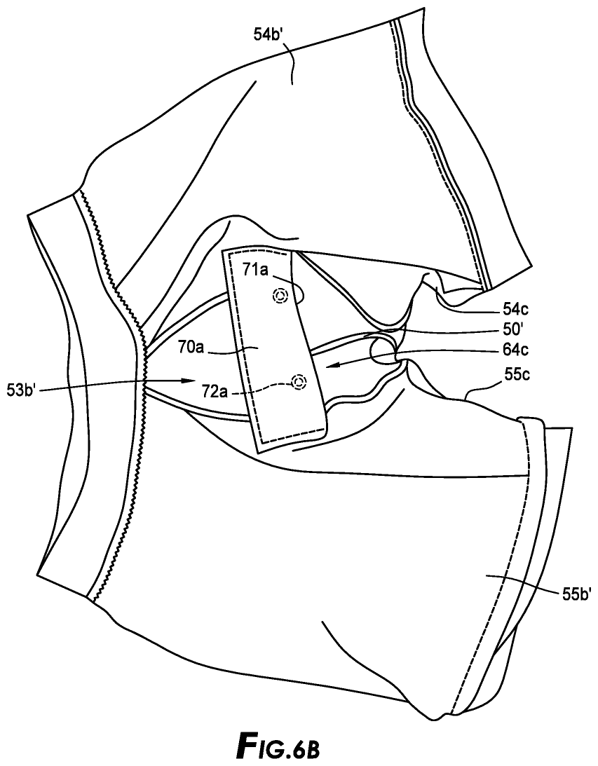

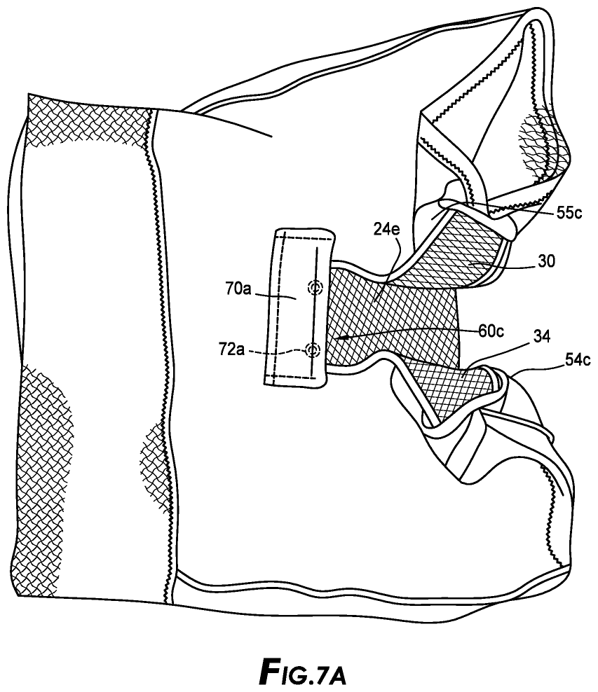

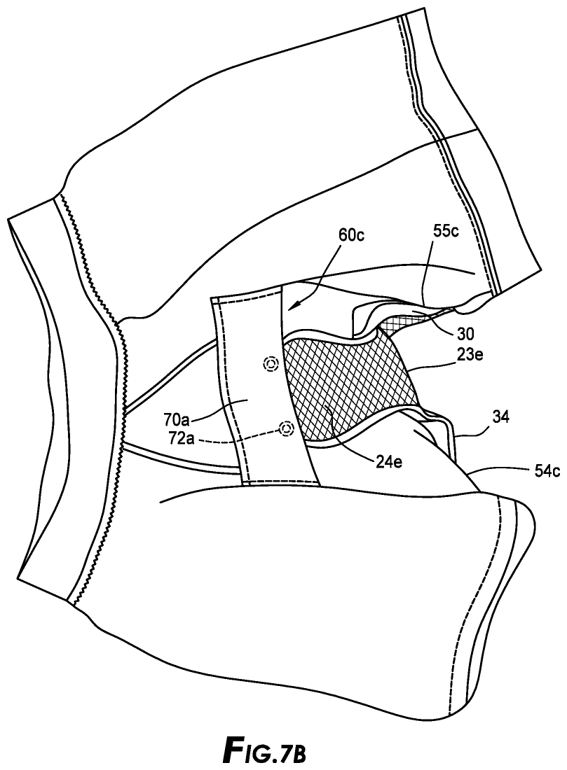

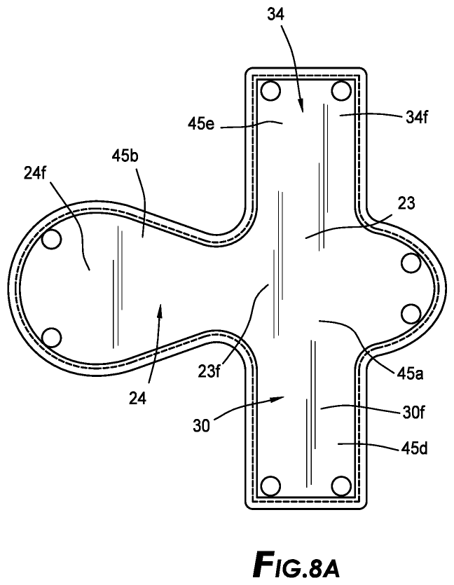

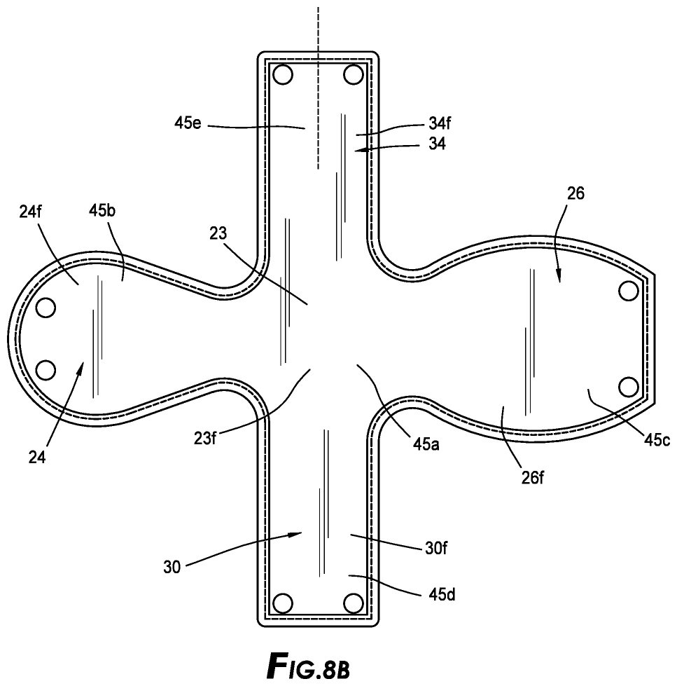

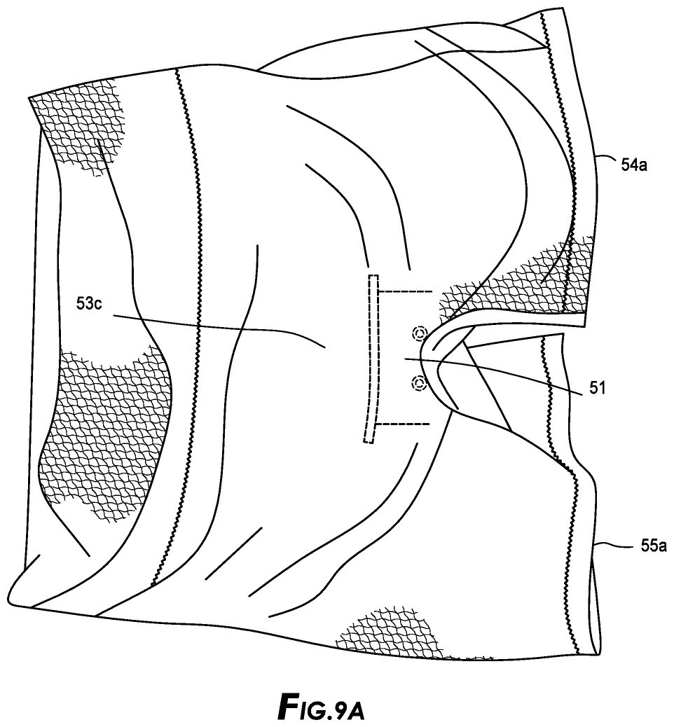

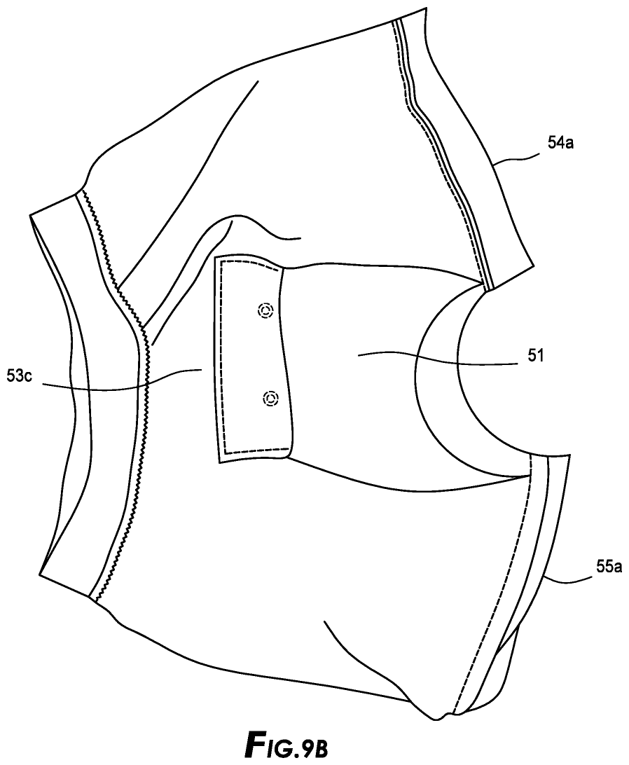

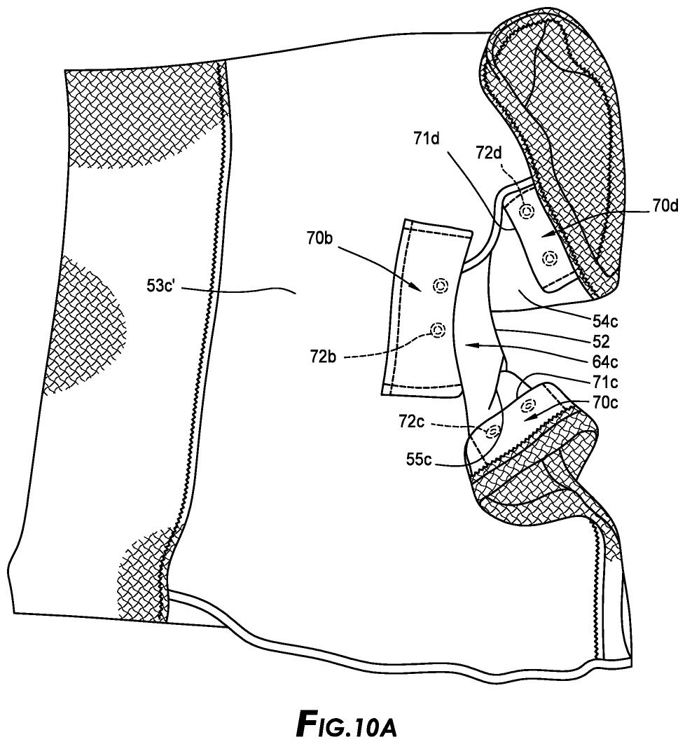

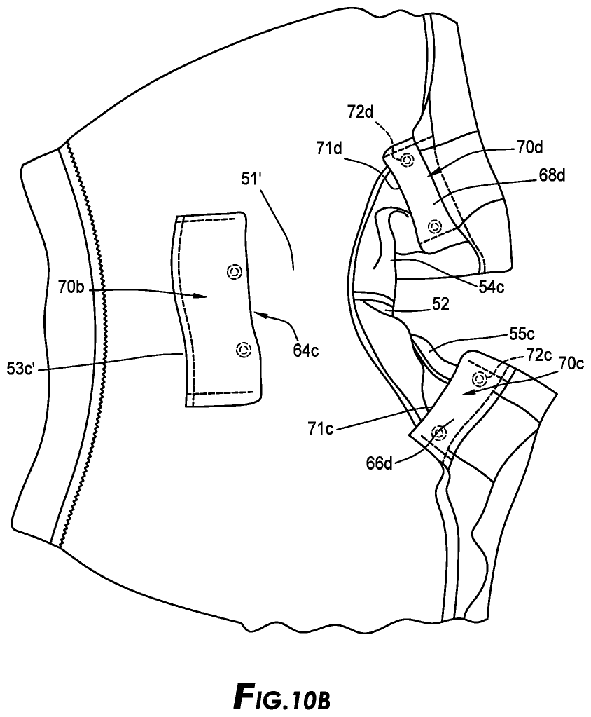

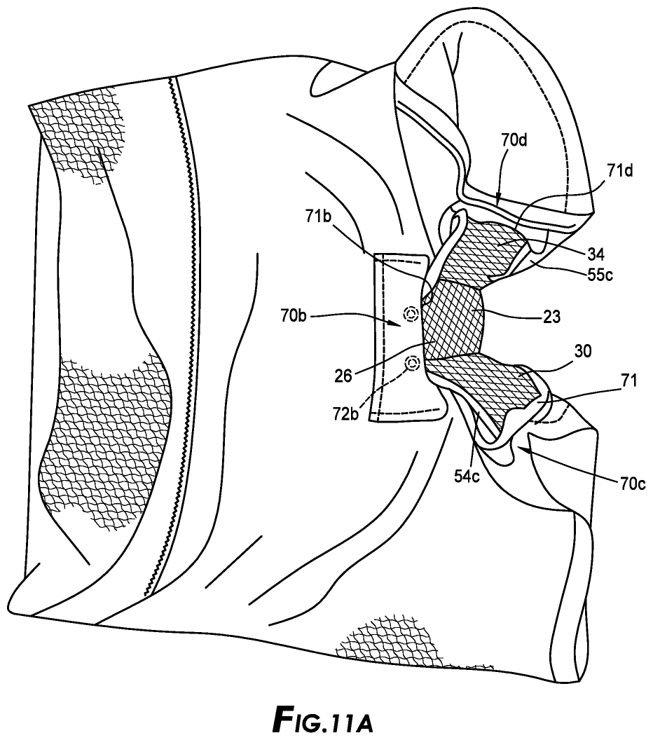

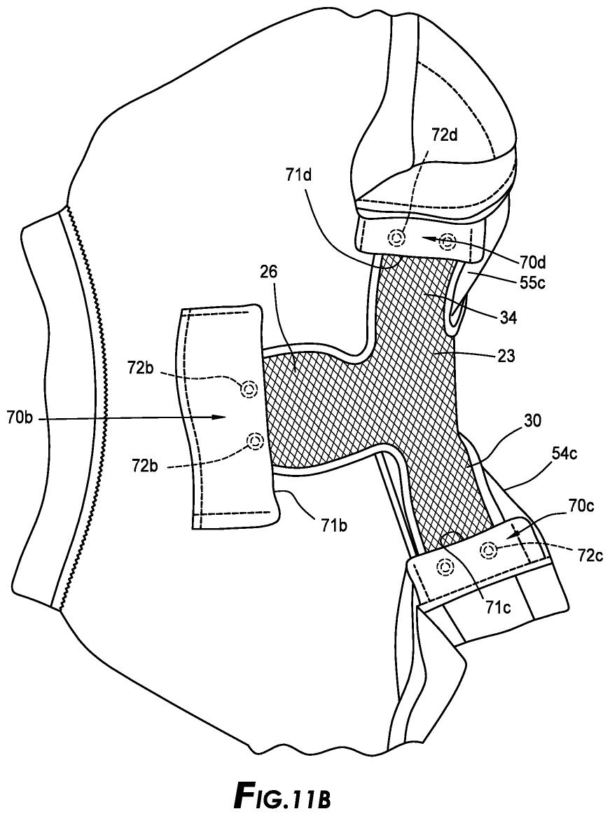

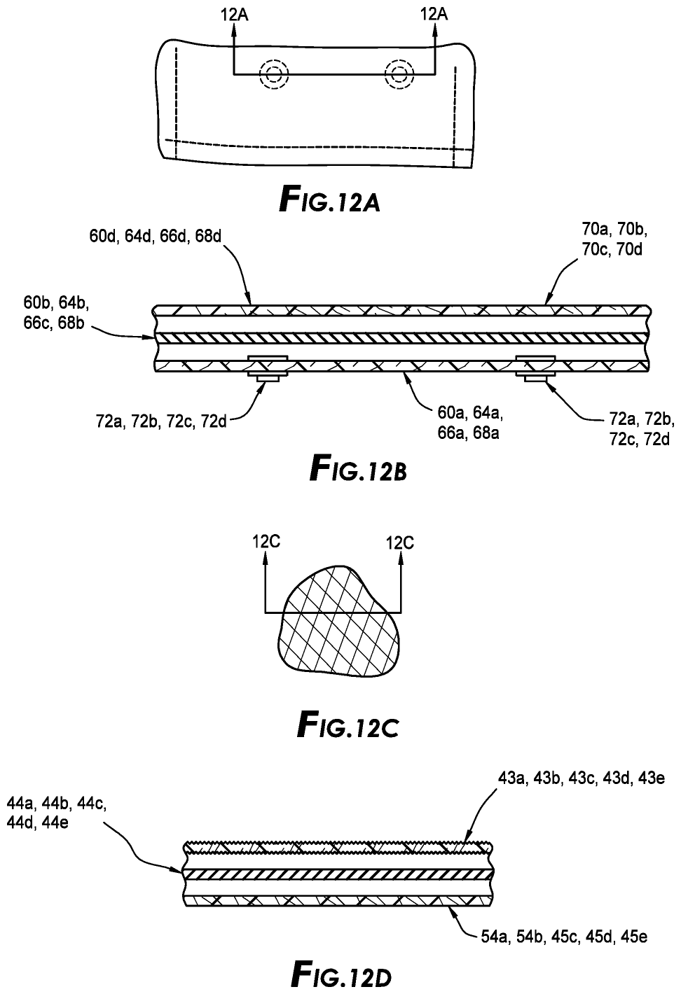

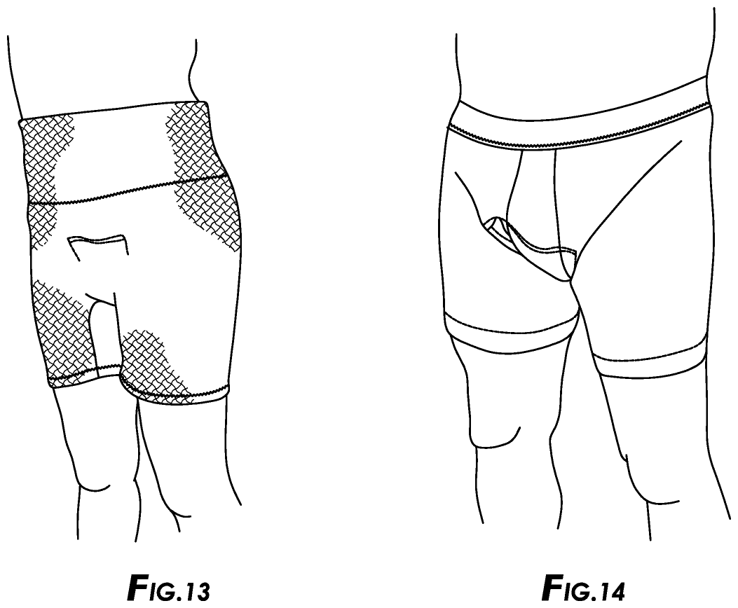

In more detail the female first extension (24), FIG. 1 has a maximum length measured from the first extension proximal end to the distal extend along the first line (25) extending along the first axis (28) of 4⅝ inches. The male first extension (24), FIG. 2, has a maximal length measured from the second extension proximal end to the distal end along the first line (25) extending along the first axis (28) of 4⅝ inches.

The first female extension (24), FIG. 1 has a width measured from the extension first side to the extension second side along a straight line extending perpendicular to the first axis (28) and taken at the midpoint of the extension's length measured from the extension's proximal end to the distal end along the first axis of 3⅝ inches. The first male extension (25), FIG. 2 has a width measured from the extension first side to the extension second side along a straight line extending perpendicular to the first axis and taken at the midpoint of the extension's length measured from the extension's proximal end to the distal end along the first axis of 3⅝ inches.

In more detail the second female extension (26), FIG. 1 has a maximum length measured from the second extension proximal end to the distal extend along the second line (27) extending along the first axis of 1⅝ inches. On the other hand, the second male extension, FIG. 2 has a maximal length measured from the second extension proximal end to the distal end along the second line (27) extending along the first axis of 4⅝ inches.

The second female extension (26), FIG. 1, has a width measured from the extension first side to the extension second side along a straight line extending perpendicular to the first axis and taken at the midpoint of extension's length measured from the extension's proximal end to the distal end along the first axis of 2⅜ inches. The second male extension (26), FIG. 2, has a width measured from the extension first side to the extension second side along a straight line extending perpendicular to the first axis and taken at the midpoint of extension's length measured from the extension's proximal end to the distal end along the first axis of 3⅞ inches.

The third (30) and fourth (34) female extensions, FIG. 1, each have a width measured from each extension first side to each extension second side along a straight line perpendicular to the second axis (41) and taken at the midpoint of each extension's length measured from each extension's proximal end to the distal end of 2½ inches. The third and fourth extension have a length measured from each extension proximal end to its distal end along a straight line extending along the second axis of 3¼ inches.

The third (30) and fourth (34) male extensions, FIG. 2 have a width measured from each extensions first side to each extensions second side along a straight line perpendicular to the second axis and taken at the midpoint of each extension's length measured from each extension's proximal end to the distal end of 2½ inches. The third and fourth extension have a length measured from each extension proximal end to its distal end along a straight line extending along the second axis of 4¼ inches.

As can be easily understood from the foregoing, the basic concepts of the present invention may be embodied in a variety of ways. The invention involves numerous and varied embodiments of a moisture absorbent undergarment and methods for making and using such moisture absorbent undergarment including the best mode. For example all of the features and structure can be medical grade.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “liner” should be understood to encompass disclosure of the act of “lining”—whether explicitly discussed or not—and, conversely, were there effectively disclosure of the act of “lining”, such a disclosure should be understood to encompass disclosure of a “liner” and even a “means for lining.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each of the moisture absorbent undergarments herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1. A device for absorbing incontinently passed urine or other moisture passed at the crotch, underside, buttocks, and/or inner thigh area of a user, said device comprising:
an intersection portion of a liner, said liner comprises padding;
a first extension of said liner extending away from said liner intersection portion and connected thereto, said first extension having a first facing surface, a second facing surface, a proximal end at the intersection portion, a distal end spaced from said proximal end a distance;
a second extension of said liner extending away from said liner intersection portion and connected thereto, said second extension having a first facing surface, a second facing surface a proximal end at the intersection portion, a distal end spaced from said proximal end a distance;
a first internal surface delimiting a first pocket interior of a first pocket;
a second internal surface delimiting a second pocket interior of a second pocket; and wherein;
said first facing surface of said first extension disposed opposite said first internal surface; and
said first facing surface of said second extension disposed opposite said second internal surface.

2. The device for absorbing incontinently passed urine or other moisture passed at the crotch, underside, buttocks, and/or inner thigh area of a user of claim 1, said device further comprising:
a first interior garment portion further delimiting said first pocket interior; said first extension disposed in said first pocket interior; and
a second interior garment portion further delimiting said second pocket interior, said second extension disposed in said second pocket interior.

3. The device for absorbing incontinently passed urine or other moisture passed at the crotch, underside, buttocks, and/or inner thigh are of a user of claim 2, said device further comprising:
a third extension of said liner extending away from said liner intersection portion and connected thereto, said third extension having a first facing surface, a second facing surface, a proximal end at the intersection portion, a distal end spaced from said proximal end a distance;
a fourth extension of said liner extending away from said liner intersection portion and connected thereto, said fourth extension having a first facing surface, a second facing surface, a proximal end at the intersection portion, a distal end spaced from said proximal end a distance;
a third internal surface delimiting a third pocket interior of a third pocket;
a fourth internal surface delimiting a fourth pocket interior of a fourth pocket; and wherein;
said first facing surface of said third extension disposed opposite said third internal surface; and
said first facing surface of said fourth extension disposed opposite said fourth internal surface.

4. The device for absorbing incontinently passed urine or other moisture passed at the crotch, underside, buttocks, and/or inner thigh area of a user of claim 3, said device further comprising:
a third interior garment portion further delimiting said third pocket interior, said third extension disposed in said third pocket interior; and
a fourth interior garment portion further delimiting said second pocket interior, said fourth extension disposed in said fourth pocket interior.

5. The device for absorbing incontinently passed urine or other moisture passed at the crotch, underside, buttocks, and/or inner thigh area of a user of claim 4, wherein:
said first internal surface delimiting said first pocket interior of said first pocket formed from first pocket structure; and
said second internal surface delimiting said second pocket interior of said second pocket formed from second pocket structure.

6. The device for absorbing incontinently passed urine or other moisture passed at the crotch, underside, buttocks, and/or inner thigh area of a user of claim 5, wherein:
said third internal surface delimiting said third pocket interior of said third pocket formed from third pocket structure; and
said fourth internal surface delimiting said fourth pocket interior of said fourth pocket formed from fourth pocket structure.

7. The device for absorbing incontinently passed urine or other moisture passed at the crotch, underside, buttocks, and/or inner thigh area of a user of claim 6, further comprising:
first coupling structure on said first extension wherein said first coupling structure releasably couples said first extension to said first pocket structure; and
second coupling structure on said second extension wherein said second coupling structure couples said second extension to said second pocket structure.

8. The device for absorbing incontinently passed urine or other moisture passed at the crotch, underside, buttocks, and/or inner thigh area of a user of claim 7, further comprising:
third coupling structure on said third extension wherein said third coupling structure releasably couples said third extension to said third pocket structure; and
fourth coupling structure on said fourth extension wherein said fourth coupling structure releasably couples said fourth extension to said fourth pocket structure.

9. The device for absorbing incontinently passed urine or other moisture passed at the crotch, underside, buttocks, and/or inner thigh area of a user of claim 8 wherein said first, second, third and fourth coupling structures all comprise a same type of coupling structure.

10. The device for absorbing incontinently passed urine or other liquid passed at the crotch area of a user of claim 9 wherein said same type of coupling structure includes a snap portion.

11. The device for absorbing incontinently passed urine or other liquid passed at the crotch area of a user of claim 10 wherein:
said first, second, third and fourth pocket structures each comprise a coupling structure, and wherein;
the coupling structure of said first pocket structure releasably mates with the first coupling structure of said first extension;
the coupling structure of said second pocket structure releasably mates with the second coupling structure on said second extension;
the coupling structure of said third pocket structure releasably mates with the third coupling structure on said third extension; and
the coupling structure of said fourth pocket structure releasably mates with the fourth coupling structure on said fourth extension.

12. The device for absorbing incontinently passed urine or other moisture passed at the crotch, underside, buttocks, and/or inner thigh area of a user of claim 11 wherein:
said first interior garment portion further delimiting said first pocket interior includes an interior of an anterior torso section; said second facing surface of said first extension opposite said interior of the anterior torso section; and
said second interior garment portion further delimiting said second pocket interior includes an interior torso section; said second facing surface of said second extension opposite said interior torso section.

13. The device for absorbing incontinently passed urine or other moisture passed at the crotch, underside, buttocks, and/or inner thigh area of a user of claim 12 wherein:
said third interior garment portion further delimiting said third pocket interior includes a first inner thigh portion of a first interior thigh section; said second facing surface of said third extension extends along said first inner thigh portion; said second facing surface opposite the interior thigh portion; and
said fourth interior garment portion further delimiting said fourth pocket interior includes a second inner thigh portion of a first interior thigh section; said second facing surface of said fourth extension extends along said first inner thigh portion; said second facing surface opposite the second interior thigh portion.

14. A liner comprising padding for absorbing incontinently passed urine or other moisture passed at the crotch, underside, buttocks, and/or inner thigh area of a user, said liner comprising:
an intersection portion;
a first extension extending away from said liner intersection portion and connected thereto, said first extension having a first facing surface, a second facing surface, a proximal end at the intersection portion, a distal end spaced from said proximal end a distance, said distance measured along a first line traversing through first structure of the first extension wherein the first structure extends from the first extension proximal end to the first extension distal end, and the first structure and first line between a first extension first side and a first extension second side;
a second extension extending away from said liner intersection portion and connected thereto, said second extension having a first facing surface, a second facing surface a proximal end at the intersection portion, a distal end spaced from said proximal end a distance measured along a second line traversing through second structure of the second extension, wherein the second structure extends from the second extension proximal end to the second extension distal end, and the structure and second line between a second extension first side and a second extension second;
a third extension of said liner extending away from said liner intersection portion and connected thereto, said third extension having a first facing surface, a second facing surface, a proximal end at the intersection portion, a distal end spaced from said proximal end a distance measured along a third line traversing through third structure of the third extension, wherein the third structure extends from the third extension proximal end to the third extension distal end, and the structure and third line between a third extension first side and a third extension second side; and
a fourth extension of said liner extending away from said liner intersection portion and connected thereto, said fourth extension having a first facing surface, a second facing surface, a proximal end at the intersection portion, a distal end spaced from said proximal end a distance measured along a fourth line traversing through fourth structure of the fourth extension, wherein the fourth structure extends from the fourth extension proximal end to the fourth extension distal end, and the structure and fourth line between a fourth extension first side and a fourth extension second side; and wherein:
said first extension inserts into a first pocket interior of a first pocket of a garment;
said second extension inserts into a second pocket interior of a second pocket of said garment;
said third extension inserts into a third pocket interior of a third pocket of said garment; and
said fourth extension inserts into a fourth pocket interior of a fourth pocket of said garment.

15. The liner of claim 14, said liner further comprising:
a first coupling structure connected to said first extension and releasably couples to a first interior garment portion;
a second coupling structure connected to said second extension and releasably couples to a second interior garment portion;
a third coupling structure connected to said third extension and releasably couples to a third interior garment portion; and
a fourth coupling structure connected to said fourth extension and releasably couples to a fourth interior garment portion.

16. The liner of claim 15, wherein:
a first interior garment portion forming a portion of a first pocket structure, said first pocket structure delimiting said first pocket interior of said first pocket, said first coupling structure connected to said first extension couples to a coupling at said first pocket structure;
a second interior garment portion forming a portion of a second pocket structure, said second pocket structure delimiting said second pocket interior of said second pocket structure, said second coupling structure connected to said second extension couples to a coupling at said second pocket structure;
a third interior garment portion forming a portion of a third pocket structure, said third pocket structure delimiting said third pocket interior of said third pocket, said third coupling structure connected to said third extension couples to a coupling at said third pocket structure; and
a fourth interior garment portion forming a portion of a fourth pocket structure, said fourth pocket structure delimiting said fourth pocket interior of said fourth pocket, said fourth coupling structure connected to said fourth extension couples to a coupling at said fourth pocket structure.

17. The liner of claim 16, wherein:
said first extension when inserted into said first pocket interior of said first pocket structure of said garment resides opposite an interior garment crotch portion of an interior garment anterior torso section;
said second extension when inserted into said second pocket interior of said second pocket structure of said garment resides opposite an interior posterior torso section;
said third extension when inserted into said third pocket interior of said third pocket structure of said garment resides opposite a first inner thigh portion of said garment; and
said fourth extension when inserted into said fourth pocket interior of said fourth pocket structure of said garment resides opposite a second inner thigh portion of said garment.

18. A liner for absorbing incontinently passed urine or other moisture passed at the crotch, underside, buttocks, and/or inner thigh area of a user comprising:
an intersection portion;
a first extension extending away from said liner intersection portion and connected thereto, said first extension having a first facing surface, a second facing surface, a proximal end at the intersection portion, a distal end spaced from said proximal end a distance, said distance measured along a first line traversing through first structure of the first extension wherein the first structure extends from the first extension proximal end to the first extension distal end, and the first structure and first line between a first extension first side and a first extension second side;
a second extension extending away from said liner intersection portion and connected thereto, said second extension having a first facing surface, a second facing surface a proximal end at the intersection portion, a distal end spaced from said proximal end a distance measured along a second line traversing through second structure of the second extension, wherein the second structure extends from the second extension proximal end to the second extension distal end, and the structure and second line between a second extension first side and a second extension second;
a third extension of said liner extending away from said liner intersection portion and connected thereto, said third extension having a first facing surface, a second facing surface, a proximal end at the intersection portion, a distal end spaced from said proximal end a distance measured along a third line traversing through third structure of the third extension, wherein the third structure extends from the third extension proximal end to the third extension distal end, and the structure and third line between a third extension first side and a third extension second side; and
a fourth extension of said liner extending away from said liner intersection portion and connected thereto, said fourth extension having a first facing surface, a second facing surface, a proximal end at the intersection portion, a distal end spaced from said proximal end a distance measured along a fourth line traversing through fourth structure of the fourth extension, wherein the fourth structure extends from the fourth extension proximal end to the fourth extension distal end, and the structure and fourth line between a fourth extension first side and a fourth extension second side; and wherein:
an installed orientation, wherein when in said installed orientation:
said first line comprises a first curve, the first curve extends along z, y coordinates of an x;−x, y;−y, z;−z coordinate system, said y, z coordinates change along the first curve;
said second line comprises a second curve, the second curve extends along z,−y coordinates of an x;−x, y;−y, z;−z coordinate system, said −y, z coordinates change along the second curve;
said third line comprises a third curve, the third curve extends along x,−z coordinates of an x;−x, y;−y, z;−z coordinate system, said x, −z coordinates change along the third curve; and
fourth line comprises a fourth curve, the fourth curve extends along −x,−z coordinates of an x;−x, y;−y, z;−z coordinate system, said −x, −z coordinates change along the fourth curve.

19. The liner of claim 18, further comprising:
a first layer comprising liquid absorbent material;
a second layer comprising liquid impermeable, resistant or repellent material; and
a third layer comprising material, said material is a same material as the interior garment posterior torso section.

20-33. (canceled)
